(19) United States
(12) Patent Application Publication
(10) Pub. No.: US 2020/0325536 A1
(43) Pub. Date: Oct. 15, 2020

(54) DNA MARKER KIT AND METHOD FOR PREPARING THE SAME

(71) Applicant: Wuhan Advance Biological Technology Co., Ltd., Wuhan (CN)

(72) Inventors: Yang LI, Wuhan (CN); Yang LI, Wuhan (CN)

(21) Appl. No.: 16/914,769

(22) Filed: Jun. 29, 2020

Related U.S. Application Data
(63) Continuation-in-part of application No. PCT/CN2018/124673, filed on Dec. 28, 2018.

(30) Foreign Application Priority Data
Dec. 29, 2017 (CN) 201711486878.0

(51) Int. Cl. C12Q 1/6876; C12Q 1/6806

(52) U.S. Cl. CPC C12Q 1/6876; C12Q 2600/166; C12Q 1/6806

(57) ABSTRACT

A DNA marker kit including a first DNA marker ranging in size from 100-1000 bp, a second DNA marker ranging in size from 1500-6000 bp, and a third DNA marker ranging in size from 5000-20000 bp. Each of the three DNA markers includes 5-6 bands.

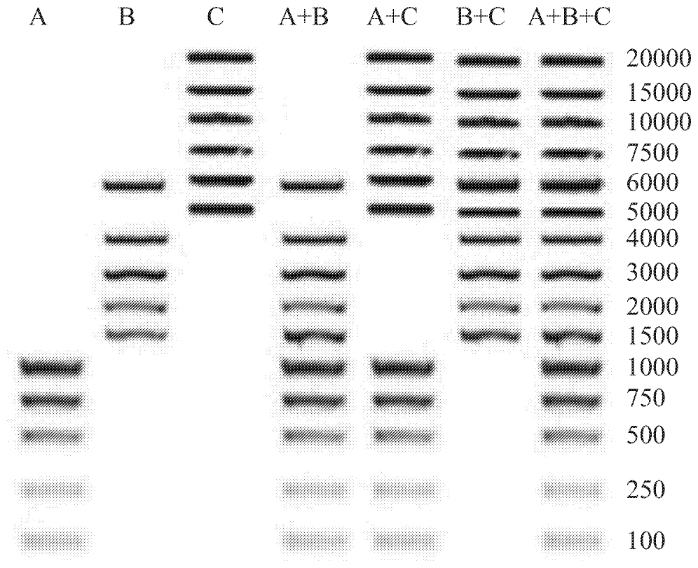

### CROSS-REFERENCE TO RELATED APPLICATIONS

This application is a continuation-in-part of International Patent Application No. PCT/CN2018/124673 with an international filing date of Dec. 28, 2018, designating the United States, now pending, and further claims foreign priority benefits to Chinese Patent Application No. 201711486878.0 filed Dec. 29, 2017. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of molecular biology and genetic engineering, and more particularly to a DNA marker kit comprising a variable number of bands and a method of preparing the kit.

DNA markers are a set of standards used for determining the approximate molecular weight of a nucleic acid fragment run on an electrophoresis gel, according to the principle that molecular weight is inversely proportional to the migration rate through a gel matrix.

Known DNA markers comprise multiple bands to mark different DNA molecules having different molecular weights. When a DNA sample to be tested comprises only one band, marking the DNA sample with the DNA markers comprising multiple bands is a waste. In addition, the DNA markers comprising multiple bands are costly.

### SUMMARY

The disclosure provides a DNA marker kit comprising a first DNA marker ranging in size from 100-1000 bp, a second DNA marker ranging in size from 1500-6000 bp, and a third DNA marker ranging in size from 5000-20000 bp. Each of the three DNA markers comprises 5-6 bands. In use, the DNA marker kit consists of one of the three DNA markers, or a combination thereof.

The first DNA marker comprises five bands with sizes of 100 bp, 250 bp, 500 bp, 750 bp and 1000 bp, respectively. The second DNA marker comprises five bands with sizes of 1500 bp, 2000 bp, 3000 bp, 4000 bp and 7500 bp, respectively. The third DNA marker comprises six bands with sizes of 5000 bp, 6000 bp, 7500 bp, 10000 bp, 15000 bp, and 20000 bp, respectively. When the second DNA marker and the third DNA marker are used in combination, the overlapped 6000 bp band is 2 times brighter than a single 6000 bp band, which can be used for quantification of nucleic acids.

The 750 bp and the 1000 bp of the first DNA marker are respectively 1.5 and 2.0 times brighter than other bands, which is used for quantification of nucleic acids.

The disclosure also provides a method for preparing the DNA marker kit, the method comprising:

1) constructing a plurality of plasmids containing DNA fragments of interest, respectively; introducing the plurality of plasmids containing DNA fragments of interest into host bacteria, respectively; and amplifying the plurality of plasmids in the host bacteria; and

2) isolating the plurality of plasmids amplified from the host bacteria, and cleaving the plurality of plasmids at specific recognition sites with restriction enzymes thereby obtaining the DNA fragments of interest; and mixing the DNA fragments of interest to obtain the DNA marker kit.

Further, the host bacteria in 1) is Escherichia coli (E. coli).

In 1), constructing the plurality of plasmids containing DNA fragments of interest comprises:

a) designing a plurality of plasmids comprising the DNA fragments required for construction of the DNA marker kit, such that the DNA fragments each comprise a single restriction site identical to another, and the single restriction site is recognizable by a restriction enzyme selected from EcoRI, Xmn I, BamH I and Hind III;

b) preparing a plurality of precursor fragments of the DNA fragments; adding a first restriction site and a second restriction site different from the single restriction site to a 5′ end and a 3′ end of each of the precursor fragments, respectively, where the first restriction site on the 5′ end is different from the second restriction site on the 3′ end of each of the precursor fragments, and the second restriction site on the 3′ end of a first precursor fragment is the same as the first restriction site on the 5′ end of a second precursor fragment next to the first precursor fragment;

c) ligating sequentially the precursor fragments to form a plurality of recombinant DNAs; amplifying the plurality of recombinant DNAs, respectively, in the presence of an upstream primer of a forefront DNA fragment and a downstream primer of a rearmost DNA fragment of each of the plurality of recombinant DNAs, where a number of single restriction sites of each recombinant DNA is equal to a number of the DNA fragments for construction of the DNA marker kit; and

d) introducing the plurality of recombinant DNAs into a plurality of vectors, respectively, thereby forming the plurality of plasmids.

Further, c) is implemented as follows: repeatedly amplifying, by PCR, the plurality of recombinant DNAs comprising the precursor fragments.

The following advantages are associated with the DNA marker kit and a preparation method thereof of the disclosure. The DNA marker comprises a first DNA marker ranging in size from 100-1000 bp, a second DNA marker ranging in size from 1500-6000 bp, and a third DNA marker ranging in size from 5000-20000 bp. Each of the three DNA markers comprises only a few bands. The bands of the three DNA markers can be used in combination. The three DNA markers can be used separately to determine the size of a particular DNA fragment, and also can be used in combination to form a DNA marker to determine the size of a group of DNA fragments ranging in different sizes. The DNA marker of the disclosure contains a total of 15 bright and clear bands.

### BRIEF DESCRIPTION OF THE DRAWINGS

The invention is described hereinbelow with reference to accompanying drawings, in which the sole FIGURE is an agarose gel electrophoresis image of three DNA markers and combinations thereof.

### DETAILED DESCRIPTION

To further illustrate the disclosure, embodiments detailing a DNA marker kit and a preparation method thereof are described below. It should be noted that the following embodiments are intended to describe and not to limit the disclosure.

The DNA marker kit comprises a first DNA marker, a second DNA marker, and a third DNA marker. The first DNA marker comprises five bands with sizes of 100 bp, 250 bp, 500 bp, 750 bp and 1000 bp, respectively. The second DNA marker comprises five bands with sizes of 1500 bp, 2000 bp, 3000 bp, 4000 bp and 7500 bp, respectively. The third DNA marker comprises six bands with sizes of 5000 bp, 6000 bp, 7500 bp, 10000 bp, 15000 bp, and 20000 bp, respectively.

The preparation of the DNA marker kit is detailed as follows:

1) Vector Design

In accordance with the preparation method of the disclosure, the cloning vector pMD19-Tsimple was employed as a vector which had an ampicillin resistance gene. BamH I was selected to perform complete digestion of recombinant DNA to prepare the DNA marker kit. The 100 bp DNA fragment contained no restriction sites at 5′ end thereof, but a Hind III restriction site at 3′ end thereof. The 250 bp DNA fragment contained a Hind III restriction site at 5′ end thereof, and an Xmn I restriction sites at 3′ end thereof. The 250 bp DNA fragment contained an Xmn I restriction site at 5′ end thereof, and an EcoRI restriction sites at 3′ end thereof. The 500 bp DNA fragment contained an EcoRI restriction site at 5′ end thereof, and an Xmn I restriction sites at 3′ end thereof. The 750 bp DNA fragment contained an Xmn I restriction site at 5′ end thereof, and an EcoRI restriction sites at 3′ end thereof. The 1000 bp DNA fragment contained an EcoRI restriction site at 5′ end thereof, and a Bam I restriction sites at 3′ end thereof. A Bam I restriction site was added downstream the restriction site at 5′ end of each of the DNA fragments except for the 100 bp DNA fragment.

2) Preparation of DNA Precursor Fragments

Primers for DNA synthesis were designed according to the sequences of the DNA fragments of different sizes. Genome of phage λ was used as a template to amplify the 100 bp DNA fragment. The amplified 100 bp DNA fragment was digested with Hind III restriction enzyme and recovered. The restriction sites at 3′ end of the DNA fragments were digested with restriction enzymes to obtain the DNA precursor fragments.

3) Assembly of DNA Precursor Fragments

The DNA precursor fragments in 2) were ligated together in a specific ratio to form a recombinant DNA, wherein the specific ratio was as follows:

First DNA marker: 100 bp:250 bp:500 bp:750 bp:1000 bp=1:1:1:1.5:2.0

Second DNA marker: 1500 bp:2000 bp:3000 bp:4000 bp:6000 bp=1:1:1:1:1

Third DNA marker: 5000 bp:6000 bp:7500 bp:10000 bp:15000 bp:20000 bp=1:1:1:1:1:1

4. Vector Assembly

The recombinant DNA in 3) was inserted into the vector pCUGIBAC1. After transformation positive clones were screened, from which the plasmid of interest was isolated.

5. Preparation of DNA Marker Kit

The plasmid of interest was introduced into and expressed by E. coli, followed by PCR amplification. The amplified plasmid of interest was extracted by lysis method used in combination with column-based DNA purification. The plasmid of interest was digested with Bam I to obtain the DNA fragments of interest and the vector pCUGIBAC1. The reaction system was as follows: 150 μL of the plasmid of interest of the first/second/third DNA marker, 30 μL of Bam I, 50 μL of a buffer, and 170 μL of ddH₂O; the reaction conditions were as follows: the reaction system was incubated at 30° C. for 6 hours. After 6 hours, the recombinant DNA was recovered by using an agarose gel column. The recovered recombinant DNA was digested with EcoRI and Hind. The reaction system was as follows: 250 μL of the recombinant DNA of the first/second/third DNA marker, 25 μL of EcoRI, 25 μL of Hind III, 60 μL of Hind III, and 140 μL of ddH₂O; the reaction conditions were as follows: the reaction system was incubated at 30° C. for 6 hours. After 6 hours, the buffer was dispersed into 9 volumes of each of the three systems to stop the enzyme digestion and reduce the chances of the three systems to interact with each other after mixing. The three DNA markers can be used to identify the size of a DNA fragment that runs on a gel during electrophoresis.

The three DNA markers and combinations thereof were observed using agarose gel electrophoresis. As shown in the sole FIGURE, the bands were separated according to the size gradually increasing from top to bottom. The 750 bp and the 1000 bp of the first DNA marker were respectively 1.5 and 2.0 times brighter than other bands. When the second DNA marker and the third DNA marker were used in combination, the completely overlapped 6000 bp band was 2 times brighter than a single 6000 bp band, which can be used for quantification of nucleic acids. The bands used in combination were widely available in various ranges of sizes of nucleic acids.

It will be obvious to those skilled in the art that changes and modifications may be made, and therefore, the aim in the appended claims is to cover all such changes and modifications.

What is claimed is:

1. A DNA marker kit, comprising:
a first DNA marker ranging in size from 100-1000 bp;
a second DNA marker ranging in size from 1500-6000 bp; and
a third DNA marker ranging in size from 5000-20000 bp;
wherein:
the first DNA marker, the second DNA marker, and the three DNA markers each comprise 5-6 bands.

2. The DNA marker kit of claim 1, wherein
the first DNA marker comprises five bands with sizes of 100 bp, 250 bp, 500 bp, 750 bp and 1000 bp, respectively;
the second DNA marker comprises five bands with sizes of 1500 bp, 2000 bp, 3000 bp, 4000 bp and 7500 bp, respectively; and
the third DNA marker comprises six bands with sizes of 5000 bp, 6000 bp, 7500 bp, 10000 bp, 15000 bp, and 20000 bp, respectively.

3. A method for preparing the DNA marker kit of claim 1, the method comprising:
1) constructing a plurality of plasmids containing DNA fragments of interest, respectively; introducing the plurality of plasmids containing DNA fragments of interest into host bacteria, respectively; and amplifying the plurality of plasmids in the host bacteria; and
2) isolating the plurality of plasmids amplified from the host bacteria, and cleaving the plurality of plasmids at specific recognition sites with restriction enzymes thereby obtaining the DNA fragments of interest; and mixing the DNA fragments of interest to obtain the DNA marker kit.

4. The method of claim 3, wherein the host bacteria in 1) is Escherichia coli (E. coli).

5. The method of claim 3, wherein in 1), constructing the plurality of plasmids containing DNA fragments of interest comprises:
a) designing a plurality of plasmids comprising the DNA fragments required for construction of the DNA marker kit, such that the DNA fragments each comprise a single restriction site identical to another, and the single restriction site is recognizable by a restriction enzyme selected from EcoRI, Xmn I, BamH I and Hind III;
b) preparing a plurality of precursor fragments of the DNA fragments; adding a first restriction site and a second restriction site different from the single restriction site to a 5′ end and a 3′ end of each of the precursor fragments, respectively, where the first restriction site on the 5′ end is different from the second restriction site on the 3′ end of each of the precursor fragments, and the second restriction site on the 3′ end of a first precursor fragment is the same as the first restriction site on the 5′ end of a second precursor fragment next to the first precursor fragment;
c) ligating the precursor fragments to form a plurality of recombinant DNAs; amplifying the plurality of recombinant DNAs, respectively, in the presence of an upstream primer of a forefront DNA fragment and a downstream primer of a rearmost DNA fragment of each of the plurality of recombinant DNAs, wherein a number of single restriction sites of each recombinant DNA is equal to a number of the DNA fragments for construction of the DNA marker kit; and
d) introducing the plurality of recombinant DNAs into a plurality of vectors, respectively, thereby forming the plurality of plasmids.

6. The method of claim 5, wherein c) is implemented as follows: repeatedly amplifying, by PCR, the plurality of recombinant DNAs comprising the precursor fragments.
